(19) United States
(12) Patent Application Publication
(10) Pub. No.: US 2010/0256503 A1
(43) Pub. Date: Oct. 7, 2010

(54) NON-INVASIVE PHOTOELECTROGLOTTOGRAPHY METHOD AND DEVICE

(75) Inventors: Kiyoshi Honda, Paris (FR); Shinji Maeda, Paris (FR)

Correspondence Address: ARENT FOX LLP, 1050 CONNECTICUT AVENUE, N.W., SUITE 400, WASHINGTON, DC, 20036, US

(73) Assignees: CENTRE NATIONAL DE LA RECHERCHE SCIENTIFIQUE - CNRS, Paris Cedex 16 (FR); UNIVERSITE DE LA SORBONNE NOUVELLE- PARIS III, Paris (FR)

(21) Appl. No.: 12/664,562

(22) PCT Filed: Jun. 16, 2008
(86) PCT No.: PCT/FR2008/000838

§ 371 Date: Jun. 23, 2010

(30) Foreign Application Priority Data
Jun. 14, 2007 (FR) 0755757

(51) Int. Cl. A61B 6/00

(52) U.S. Cl. 600/476

(57) ABSTRACT

The aim of the invention is to provide a visualisation of glottal behaviour in different phonation conditions (voiced or unvoiced consonants, vowels) without articulatory restriction and without limits in phoneme analysis. For this purpose, the invention provides a non-invasive approach comprising the diffuse illumination of the glottis using an external light source and the collection of the light signal by means of external photodetection. In accordance with the device of the invention, a light source (10, 12) is positioned externally to the person (P) being examined, at the lateral hypopharynx (HP). A photodetector (20), masked (22) against ambient light, is positioned immediately next to the skin (11) of the person (P) being examined. Said photodetector (20) is sensitive at least to the wavelength range of the light diffused by the source (10, 12) and is connected to an amplifier (24) which amplifies the signal emitted by the photodetector which, together with a signal (26) processing step, allows successive data relating to glottis opening amplitude to be viewed.

The invention concerns a process to analyze the behavior of the larynx glottis by non invasive photoelectroglottography, as well as an equipment for implementation by diffuse light emission and detection.

The invention relates to clinical and experimental research in the domain of speech sciences—phonetics and phonology—from visualizing the behavior of phonation organs, in particular of the larynx and glottis.

Voice and therefore speech is commanded, namely, by the opening and closing of the larynx glottis, a space limited by the folds forming the vocal cords. Visualizing the variation of the state and quality of the glottis opening/closing, revealing glottis behavior, is an essential step in clinical and experimental research. The complexity of the speech organ mechanisms indeed requires combining aerodynamic, physiological, biomechanical, and acoustic data, in order to study intra- and interactive coordination between phonation organs (larynx and lungs) and articulation organs (tongue, lips, soft palate, pharynx).

Interpreting aerodynamic data can be validated by photoglottographical data that provides information on temporal variations of the glottis area. Known photoglottographical techniques use larynx illumination through fibroscopy, by means of a nasal fibroscope, and extra-pharyngeal sensor for detection.

However, photoglottography cannot provide information that represents glottis behavior because signal hindrance and distortion caused by such invasive techniques induce an uncertainty that prevents interpretation. Moreover, existing techniques can only be used to explore phonemes such as vowels /i/ and /e/ corresponding to an anterior position of the tongue of the person examined.

The invention is aimed at providing a visualization of glottis behavior in different situations of phonation (vowels, voiced or non voiced consonants) for all languages used in the world, and of voice quality (blown, cracked, etc.), without articulatory restriction and without limit in phoneme exploration.

In order to achieve this, the invention proposes a non invasive approach by diffuse illumination of the glottis from an external light source and by collecting the luminous signal through external photodetection too.

More precisely, the invention has for object a non invasive photoelectroglottography process in a zone of sound formation for a person's speech, said zone including a hypopharynx, a larynx, and a glottis bordered by vocal folds. A light, external to the person and located in immediate surroundings of the person's skin, illuminates the glottis, at a power and within a wavelength range capable to cross the person's skin by diffuse transmission passing through the hypopharynx and the larynx. A diffuse luminous signal is collected by photodetection outside the person after crossing the glottis, said signal being then amplified and processed to successively provide time dependent data from the glottis successive openings and closings, hence defining glottis opening amplitude.

Glottis opening amplitude during speech provides two types of information: on vocal folds vibration for pronouncing voiced sounds such as vowels and sounded consonants, and on vocal fold adduction/abduction enabling the distinction between silent consonants and sounded consonants. Said amplitude also represents phoneme accentuated position or not. The process can thus be used in the domain of the experimental and clinical phonetics as well as for compared study of languages, because the position of the tongue, massed forward or not, is indifferent when applying this process.

The invention also relates to equipment for applying said process. Said non invasive equipment includes a light source positioned outside the person examined over the lateral hypopharynx. It also includes a photodetector sheltered from ambient lighting and positioned in the immediate surroundings of the skin of the person examined. Such photodetector is sensitive to at least the wavelength range of the light emitted by the source, and is connected to an amplifier for the signal emitted by the photodetector that enables, through signal processing, to visualize successive data of glottis opening amplitude.

According to particular forms of embodiment:
the signal processing is realized with a recorder that delivers chronograms of the amplified signal;
the light source is a light guide linked to a temperature-isolated transmitter, or more advantageously a light emitting diode (LED), the source possibly being also directly this transmitter without guide;
the light source is a high power LED controlled by a pulse train generator (13);
ambient lighting is filtered by the photodiode amplifier circuit, using a low-pass filter;
the LED is of strong power in the order of several Watts, typically between 1 and 3 Watts, and emitting in an active wavelength range from red to infrared, in particular near infrared;
the photodetector is positioned under the glottis in the direction of light propagation;
Processing the signal collected by the photodetector provides a recording and/or a direct visualization of the data, with an oscilloscope or suitable computer-driven software;
the transmitter wavelength range is located in the red—infrared for an effective penetration of the skin;
means of stabilizing the luminous signal of the light source are provided in order to prevent signal fluctuations, notably through a stabilized direct current source;
the photodetector can be a high sensitivity and high speed response PIN type photodiode;
the light source is positioned outside the person but immediately near the skin, in the space located against the hyoid bone and thyroid cartilage, close to the upper horn of the thyroid cartilage;
the photodetector can be attached to the skin in combination with any means for absorbing or reflecting the surrounding light, in the space located against the thyroid cartilage and the cricoid cartilage, or the tracheal region.

Other characteristics and advantages of the invention will appear in the description that follows relative to a non restrictive example of embodiment, and referring to the attached figures that represent respectively:

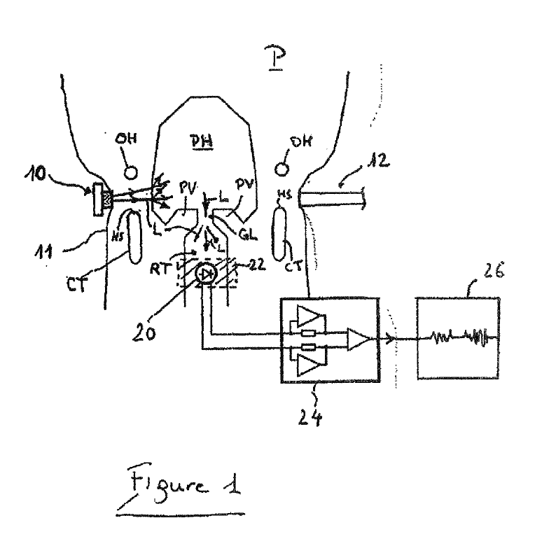

FIG. 1, a general diagram of an equipment according to the invention in operational position on the person examined appearing in cross-section;

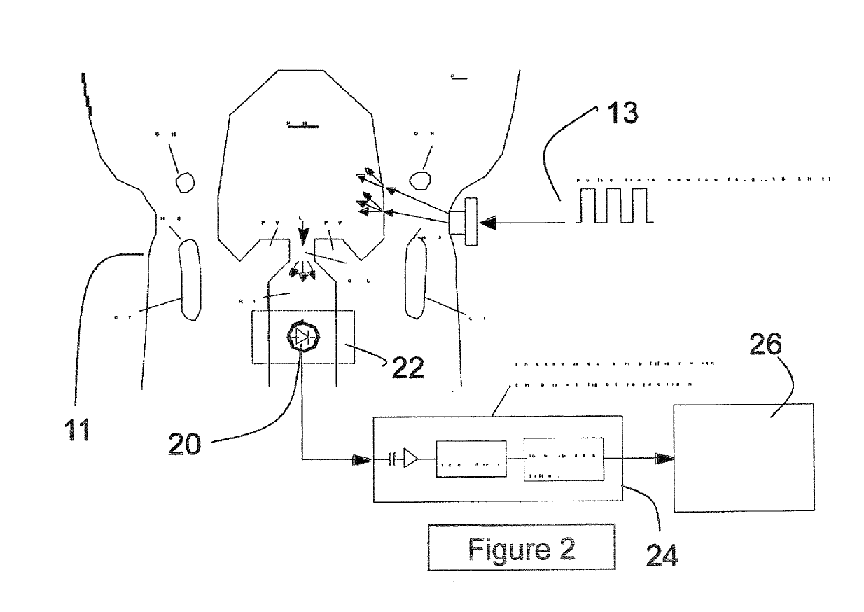

FIG. 2, a general diagram of an equipment according to another form of embodiment of the invention in operational position on the person examined appearing in cross-section; and

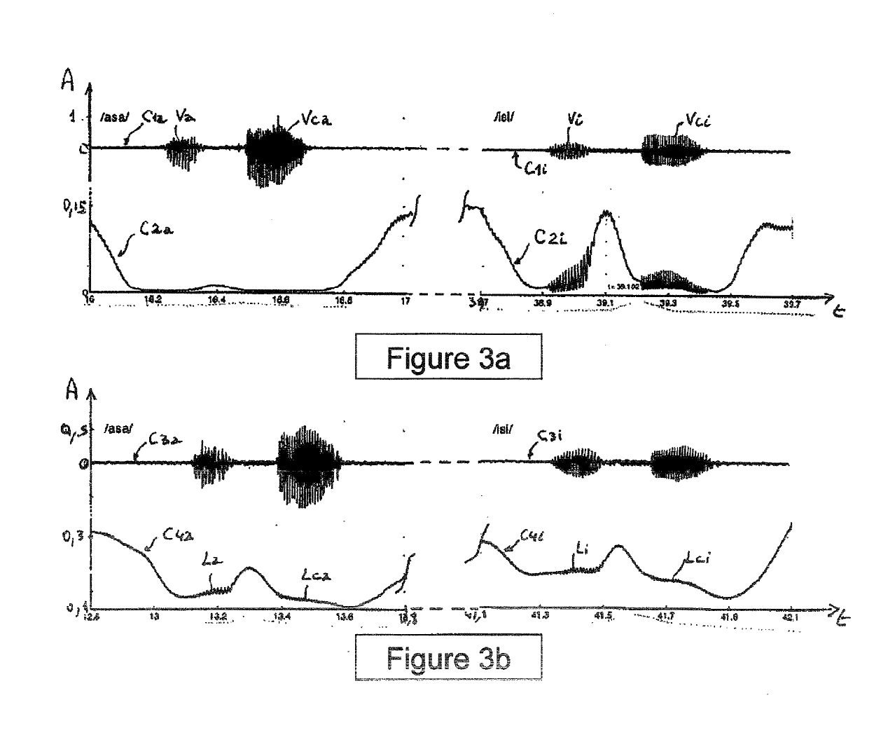

FIGS. 3a and 3b, chronograms comparing glottis amplitude signals obtained with nasal fibroscopic equipment (FIG. 3a) and an equipment according to the invention (FIG. 3b) for two types of sounds with posterior open /a/ and anterior closed /i/ vowels; simultaneous voice recordings obtained with a microphone are also represented with each chronogram.

The example of non invasive photoelectroglottography equipment according to the invention, as illustrated in FIG. 1, include a 3 watt light emitting diode 10 (hereafter LED) as light source. LED 10 is positioned on against skin 11 of person P examined, in the region of hypopharynx HP (approximately represented in FIG. 1), more precisely between hyoid bone OH and thyroid cartilage CT, close to the upper horn HS of thyroid cartilage CT. Instead of the LED, but in a similar positioning, it is possible to use a light guide 12 transporting light from a stabilized “cold” transmitter, e.g. a halogen lamp with a heat absorbing filter. The LED wavelength is in near infrared for an effective penetration of the skin.

Advantageously, means of stabilizing the light source luminous signal are provided in order to prevent signal fluctuations, notably through a stabilized direct current LED power source;

The equipment also includes, as photodetector 20, a high sensitivity and high speed response PIN type photodiode. Photodetector 20 is also positioned against the examined person's skin 11, but under the person's glottis GL if one follows the propagation direction of light L. More precisely, it is arranged in the space located in tracheal region RT. Thus, light L coming from the LED is diffused (L arrows in FIG. 1), reflected in pharynx PH, and passed through open glottis GL, bordered by vocal folds PV (forming the vocal cords).

In order to collect only the light coming from the LED, the photodetector is advantageously isolated from ambient lighting using a black absorbing mask 22.

The photodetector is most sensitive in the LED infrared emission range. The signal collected by the photodiode is transmitted to a signal amplifier 24 connected to a signal recorder 26. Said recorder is, in the example, an oscilloscope that is capable, through classic processing, of visualizing the amplitude of the signal transmitted through the opening of glottis GL. It is also possible to visualize the signal with a suitable computer-driven audio software (as shown in FIG. 2).

The example of non invasive photoelectroglottography equipment according to the invention, as illustrated in FIG. 2, uses the setup described in support of FIG. 1 but includes a high power LED as light source, controlled by a pulse train generator 13, e.g., at 10 kHz frequency. It allows the signal amplifier 24 to filter interferences due to ambient lighting while integrating a coupled photo-diode amplifier, a rectifier circuit, and a low pass filter (ambient lighting having a frequency around 1014 Hz). The skilled man will be able to adapt such filter circuit using, e.g., a lock-in amplifier.

The recorder can deliver chronograms in real time by plotting the amplitude of signal A as a function of time t as represented in FIGS. 3a and 3b. The chronograms as illustrated correspond respectively to open vowel [a] and silent consonant [s] of “asa” (pronunciation: “assa”) phoneme sounds, and to closed vowel [i] and silent consonant [s] of “isi” (pronunciation: “issi”). In the first chronograms, C1a and C1i curves indicate the audio amplitude obtained with a microphone placed in front of the person. C1a and C1i represent acoustic vibrations Va and Vi while pronouncing vowels [a] and [i], then a noise zone while pronouncing silent consonant [s] for “sa” and “si”, then vibrations Vca and Vci while pronouncing second vowel [a] and [i] of the logatomes.

Recordings, curves C2a and C2i (FIG. 3a), by fibroscopy photoglottography, were realized respectively and simultaneously with recordings C1a and C1i. Recordings C2a and C2i show the dependency of this invasive technology with the articulation of the concerned vowel, since C2a signal remains almost flat during posterior open vowel “a” and during corresponding silent consonant [s] in “sa” (posteriorized [s] articulation because of the tongue position during posterior vowel [a], by coarticulation). Only in the case of pronouncing an anterior vowel [i], “i”, and corresponding silent consonant [s] as in “si”, does signal C2i provide the responses corresponding to sequences Vi and Vci.

Other than that, recordings were realized under the same vocal expression conditions by comparing the recordings of signals formed by microphone (C3a and C3i) and by the equipment according to the invention (C4a and C4i). Recordings realized with said equipment show then that the proposed process and equipment are not dependent upon the articulation of the concerned vowel: indeed, signals C4a and C4i obtained with the non-invasive technology according to the invention present significant and measurable signal amplitudes La and Lca, then Li and Lci, whatever the type of vowels (posterior or interior) pronounced: the delivered signals are of a globally similar form for [asa] and [isi], which corresponds to physiological reality. It comes from the fact that the posteriorized position of the tongue in the case of [asa] does not mask such measurements; contrary to the recordings done by fibroscopy, as it appears in curve C2i.

So the diffused light crosses the glottis whatever the tongue position, and sensibly translates the amplitude variations of the glottis and vocal folds that participate to it: the signal depends on the successive openings and closings of the glottis. It follows that the non invasive light diffusion technology is independent of the vowel or consonant type.

Under such conditions, the glottis opening amplitude during speech provides information on vocal fold vibration for pronouncing vowels, and on vocal fold adduction/abduction permitting a distinction between silent and sounded consonants, such amplitude also translating the position accented or not of the phonemes, the tongue position massed forward or not being indifferent, as explained above.

The invention is not limited to the examples of embodiment either described or represented. It is, for example, possible to provide several LED as light source, arranged close by or symmetrically in relation to the trachea. In this case, various tilts can be applied in order to collect more light. It is also possible to provide a broad range photodetector or several photodetectors.

1. Non invasive photoelectroglottography process in a zone of sound forming of a person's speech, said zone including a hypopharynx (HP), a pharynx (PH), and a glottis (GL) bordered by vocal folds (PV), wherein it consists in a light (10, 12) external to person (P) and located in immediate surroundings of skin (11) of said person, to illuminate at a power and within a wavelength range enabling to cross the person's skin, glottis (GL) by transmission under diffuse form (L) by passing through hypopharynx (HP) and pharynx (PH), and wherein a diffuse luminous signal is collected by photodetection outside the person after crossing glottis (GL), said signal being then amplified and processed to provide data depending successively in time on the successive glottis opening and closing, thus defining a glottis opening and oscillation amplitude (A).

2. Non invasive photoelectroglottography process according to claim 1, wherein, the sound forming zone of a person's speech symmetrically including a hyoid bone (OH) and a thyroid cartilage (CT) presenting an upper horn (HS), light source (10, 12) is positioned in a space located against hyoid bone (OH) and thyroid cartilage (CT), and near upper horn (HS) of said thyroid cartilage.

3. Equipment for implementing the process according to claim 1 or 2, wherein it includes a light source (10, 12) positioned externally to person (P) examined at the level of lateral hypopharynx (HP), a photodetector (20) masked from ambient lighting and positioned in the immediate surroundings of skin (11) of person (P) under examination, said photodetector (20) being sensitive at least to the light wavelength range emitted by source (10, 12), and wherein photodetector (20) is connected to an amplifier (24) of the signal emitted by the photodetector enabling to, linked to a processing of signal (26), visualize successive data of opening amplitude (A) of the glottis.

4. Equipment according to claim 3, wherein the signal processing is realized by recorder (26) that delivers chronograms of the amplified signal.

5. Equipment according to claim 3, wherein the light source is a light guide (12) linked to a temperature-isolated transmitter, or directly to said LED type transmitter (10).

6. Equipment according to claim 3, wherein the light source is a high power LED controlled by a pulse train generator (13).

7. Equipment according to claim 3, wherein ambient lighting is filtered by the photodiode amplifier circuit, using a low-pass filter.

8. Equipment according to claim 5, wherein the transmitter is a light emitting diode (LED) of power ranging between 1 and 3 watts and emitting in a range of active wavelengths from red to infrared, in particular near infrared.

9. Equipment according to claim 3, wherein it includes an oscilloscope and/or a suitable computer-driven software enabling a recording and/or a direct visualization of the data provided by the processing of the signal collected by photodetector (20).

10. Equipment according to any claim 3, wherein luminous signal stabilization means of the light source are provided in order to prevent signal fluctuations, notably using a stabilized direct current source.

11. Equipment according to claim 3, wherein photodetector (20) is a type of high sensitivity and high speed response PIN photodiode.

12. Equipment for implementing the process according to claim 3, wherein photodetector (20) is attached to skin (11) in combination with a mask (22) absorbing or reflecting surrounding light, in the space located against thyroid cartilage (CT) and cricoid cartilage or tracheal region (RT).
